SEQUENCE LISTING (12 sequences)

SEQ ID NO: 1 - protein, 15 aa, Artificial Sequence
  Synthetic peptide
MGNNIKVTFNPDKIA

SEQ ID NO: 2 - protein, 20 aa, Artificial Sequence
  Synthetic peptide
GRKPTPPTPPLRDTHPHLTM

SEQ ID NO: 3 - protein, 23 aa, Artificial Sequence
  Synthetic peptide
DPALSPEMSPSSLLGLLAGLQVV

SEQ ID NO: 4 - protein, 20 aa, Artificial Sequence
  Synthetic peptide
YFLWTKILTIAQNLDWWCTS

SEQ ID NO: 5 - protein, 20 aa, Artificial Sequence
  Synthetic peptide
YCCCLKPTAGNCTCWPIPSS

SEQ ID NO: 6 - protein, 21 aa, Artificial Sequence
  Synthetic peptide
LSILPPFIPIFVLFFLIWVYI

SEQ ID NO: 7 - protein, 18 aa, Artificial Sequence
  Synthetic peptide
LTMKNQTFHLQGFVDGLR

SEQ ID NO: 8 - protein, 18 aa, Artificial Sequence
  Synthetic peptide
LTMKDQTFHLQGFVDGLR

SEQ ID NO: 9 - protein, 18 aa, Artificial Sequence
  Synthetic peptide
CLKPTAGNCTCWPIPSSW

SEQ ID NO: 10 - protein, 18 aa, Artificial Sequence
  Synthetic peptide
CLKPTAGDCTCWPIPSSW

SEQ ID NO: 11 - protein, 9 aa, Artificial Sequence
  Synthetic peptide
KPSDGNCTC

SEQ ID NO: 12 - protein, 9 aa, Artificial Sequence
  Synthetic peptide
KPSDGDCTC

(19) United States
(12) Patent Application Publication
(10) Pub. No.: US 2017/0136120 A1
(43) Pub. Date: May 18, 2017

(54) INDUCTION OF IMMUNE RESPONSE

(71) Applicant: Drexel University, Philadelphia, PA (US)

(72) Inventors: Timothy M. Block, Doylestown, PA (US); Anand Mehta, Lansdale, PA (US); Pamela Norton, Blue Bell, PA (US)

(21) Appl. No.: 15/420,434

(22) Filed: Jan. 31, 2017

Related U.S. Application Data
(63) Continuation of application No. 14/801,371, filed on Jul. 16, 2015.
Application No. 14/801,371 is a continuation of application No. 13/805,844, filed on Feb. 5, 2013, now abandoned.
(60) Provisional application No. 61/358,777, filed on Jun. 25, 2010.

(51) Int. Cl. A61K 39/29; C12N 7/00; A61K 31/7072; A61K 45/06

(52) U.S. Cl. CPC A61K 39/292; A61K 45/06; C12N 7/00; A61K 31/7072; C12N 2730/10134; A61K 2039/572

(57) ABSTRACT

Provided are methods and compositions that can he used to treat subjects having a viral infection by provoking an immune response using newly discovered antigens that are non-naturally occurring variations on viral glycoproteins. For example, provided are viral glycoproteins or a fragments thereof, or, DNA constructs encoding for such viral glycoproteins or fragments thereof, wherein the glycoprotein or fragment comprises a glycosylation sequon that includes a non-templated aspartic acid residue.

### CROSS-REFERENCE TO RELATED APPLICATION

This application is a continuation of U.S. Ser. No. 13/805,844, filed Feb. 5, 2013 as the National Stage of International Application No. PCT/US2011/041829, filed Jun. 24, 2011, which claims the benefit of U.S. Provisional Application No. 61/358,777 filed Jun. 25, 2010, the entire disclosures of which are incorporated herein by reference for any and all purposes.

### GOVERNMENT RIGHTS

Research leading to the disclosed invention was funded, in part, by the U.S. National Institutes of Health, Grant Nos. UO1 A1053884 to Timothy M. Block and U01A1054763 to Anand Mehta. Accordingly, the United States Government has certain rights in the invention described herein.

### TECHNICAL FIELD

The present disclosure concerns the use of pharmacological agents and/or other moieties in order to induce an immunological response to viral infection.

### BACKGROUND

Chronic infection with hepatitis B virus (HBV) is characterized by a lack of robust T cell responsiveness to viral antigens (1, 2). Indeed, an inadequate CD8+ T cell response is thought to be key to the establishment of chronicity. Typically, virus-specific CD8+ cytotoxic T lymphocytes (CTLs) are elicited by infected cells presenting virus-derived peptides by major histocompatibility complex (MHC) class I. However, poor CTL responses in chronic HBV infection are likely a consequence of multiple factors (1, 2), including viral interference with efficient processing and presentation of HBV epitopes (3). Thus, methods that can cause enhanced recognition or presentation of viral epitopes by MHC class I might be useful as therapeutic interventions and as research tools.

Viral glycoproteins represent important targets for any antiviral immune response. HBV is an enveloped virus with three glycoproteins: LHBs, MHBs and SHBs (4). In tissue culture, the HBV envelope proteins are very stable, and are degraded by proteasomes less efficiently than host proteins (5). Resistance to proteasomal degradation might contribute to HBV's refractoriness to presentation by MHC class I and even to establishment of chronicity (6). However, compared to most cellular N-glycoproteins, and even the SHBs, the MHBs protein is unusually dependent upon calnexin mediated protein folding (7, 8). Calnexin is a cellular lectin chaperone that recognizes N-glycans on nascent proteins that have been trimmed to a mono- glucose residue (9, 10). This trimming is mediated by glucosidases in the endoplasmic reticulum (ER). Inhibition of glucosidases resulted in significant and selective degradation of MHBs under conditions where most cellular glycoproteins are spared (7, 11). The sensitivity of MHBs to glucosidase inhibition was correlated with antiviral activity in animals (11).

There remains a therapeutic and investigational need for techniques that can provoke enhanced recognition or presentation of viral epitopes by the major histocompatability complex.

### SUMMARY

Provided are methods for treating a subject having a viral infection comprising administering to the subject a composition comprising a viral glycoprotein or a fragment thereof, or, a DNA construct encoding for the viral glycoprotein or fragment thereof, wherein the glycoprotein or fragment comprises a glycosylation sequon that includes a non-templated aspartic acid residue.

Also provided are viral glycoproteins or a fragments thereof, or, DNA constructs encoding for such viral glycoproteins or fragments thereof, wherein the glycoprotein or fragment comprises a glycosylation sequon that includes a non-templated aspartic acid residue. The present disclosure also relates to compositions comprising such viral glycoproteins or a fragments thereof, or, DNA constructs encoding for such viral glycoproteins or fragments thereof, and a pharmaceutically acceptable carrier.

### BRIEF DESCRIPTION OF THE DRAWINGS

FIG. 1 provides a schematic representation of the consequences of endoplasmic reticulum associated degradation-linked de-N-glycosylation,

FIG. 2 provides data demonstrating that CTLs raised against aspartic containing envelope protein epitopes recognize HBV producing cells.

FIG. 3 depicts the experimental vaccination schedule for woodchucks, and illustrates the degree of proliferation of PBMCs in response to viral antigens.

FIG. 4 provides data relating to the proliferation of PBMCs induced by viral neo-antigen in response to drug treatment.

FIG. 5 relates to the proliferation of PBMCs in response to neo-antigen vaccination.

### DETAILED DESCRIPTION OF ILLUSTRATIVE EMBODIMENTS

The present invention may be understood more readily by reference to the following detailed description taken in connection with the accompanying figures and. examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a glycoprotein” is a reference to one or more of such materials and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.”

The disclosures of each patent, patent application, and publication cited or described in this document are hereby incorporated herein by reference, in their entirety. Numbers in parenthesis correspond to the numbered list of references that is provided in the final portion of this disclosure.

Unless otherwise specified, any component, element, attribute, or step that is disclosed with respect to one aspect of the present invention (for example, the methods, peptides, proteins, DNA sequences, compositions, respectively) may apply to any other aspect of the present invention (any other of the methods, peptides, proteins, DNA sequences, compositions, respectively) that is disclosed herein.

The present disclosure demonstrates, inter alia, the pharmacological alteration of viral epitopes, including, for example, the hepatitis B virus (HBV) epitopes, presented by major histocompatibility complex (MHC) class I on infected cells. The HBV middle envelope glycoprotein MHBs maturation appears to require calnexin mediated folding. This interaction is dependent upon glucosidases in the endoplasmic reticulum. Prevention of HBV envelope protein maturation in cultured cells with glucosidase inhibitors, such as 6-O-butanoyl castanospermine and N-nonyl deoxynorjirmycin, resulted in MHBs degradation by proteasomes. The de-N-glycosylation associated with polypeptide degradation was predicted to result in conversion of asparagine residues into aspartic acid residues. This prediction was confirmed by showing that proteins, peptides, or corresponding DNA sequences that include the N-glycosylation sequons of MHBs, but with aspartic acid replacing asparagine, (a) can prime human CTLs that recognize HBV producing cells and (b) that the presentation of these envelope motifs by MHC class I is enhanced by incubation with glucosidase inhibitors. Moreover, although peripheral blood mononuclear cells isolated from woodchucks chronically infected with woodchuck hepatitis virus (WHV) and vaccinated with WHV surface antigen could be induced to recognize the natural MHBs asparagine-containing peptides, only cells isolated from glucosidase inhibitor treated animals recognized the aspartic containing peptides. These data demonstrate that pharmacological intervention with peptides or proteins with asparagine containing glycosylation sequons, with or without glucosidase inhibitors and/or antiviral agents (such as nucleoside analogs) can alter the MHBs epitopes presented. This editing of the amino acid sequence of the polypeptide therefore results in a new epitope, or “epitope” of medical significance.

Degradation of MHBs in the presence of glucosidase inhibitors was mediated by cellular proteasomes (5, 12). Proteasomal degradation products are substrates for MHC class I-mediated presentation to T cells. It was presently hypothesized that glucosidase inhibitors could selectively enhance presentation of MHBs epitopes. This prediction was confirmed in cell culture; glucosidase inhibitor treatment of target cells resulted in increased killing by peptide- specific CTLs (13). Degradation of MHBs following glucosidase inhibition might also be accompanied by de-N-glycosylation. Hydrolysis of N-linked glycan from asparagines of glycoproteins is thought to occur in the cytoplasm by the enzyme peptide: N-glycanase (PNGase) (14), resulting in conversion to aspartic acid (15, 16). Thus, de-N-glycosylation of MHBs in glucosidase-inhibited cells should be accompanied by altered polypeptide amino acid composition. It was postulated by the present inventors that such edited epitopes, or “epitopes”, could be created by pharmacological intervention with glucosidase inhibitors, and that such epitopes could be used to provoke an immune response. Although presentation of peptides containing aspartic acid in place of asparagines has been reported (17-19), the pharmacological induction of this modification would be unprecedented and have profound implications for therapy and how neo-antigens might be created. The present disclosure includes the results of such an intervention in tissue culture and in woodchucks chronically infected with woodchuck hepatitis virus (WHV), which mimics many of the immunologic features of chronic HBV infection in humans (20).

The present disclosure provides are methods for treating a subject having a viral infection (such as a chronic viral infection) comprising administering to the subject a composition comprising a viral glycoprotein or a fragment thereof, or, a DNA construct encoding for the viral glycoprotein or fragment thereof, wherein the glycoprotein or fragment comprises a glycosylation sequon that includes a non-templated aspartic acid residue.

Also provided are viral glycoproteins or a fragments thereof, or, DNA constructs encoding for such viral glycoproteins or fragments thereof, wherein the glycoprotein or fragment comprises a glycosylation sequon that includes a non-templated aspartic acid residue. The present disclosure also relates to compositions comprising such viral glycoproteins or a fragments thereof, or, DNA constructs encoding for such viral glycoproteins or fragments thereof, and a pharmaceutically acceptable carrier.

As used herein, the term “non-templated aspartic acid” residue refers to an aspartic acid residue that occurs due to de-amidation of a templated asparagine residue. Preferably, the viral glycoprotein or fragment corresponds to the naturally occurring counterparts from the virus with which the subject is infected. The virus with which the subject is infected (and to which the viral glycoprotein or fragment thereof corresponds) may be any virus having one or more envelope proteins that are sensitive to glucosidase inhibitors. Sensitivity to glucosidase inhibitors refers to a measurable prevention of de-glycosylation of the one or more viral envelope proteins. For example, the virus may be any enveloped virus, such as hepatitis B virus or hepatitis C virus. Numerous other enveloped viruses are well known among those of ordinary skill in the art, and all enveloped viruses are contemplated.

The viral glycoprotein may be an envelope protein. For example, the glycoprotein may be a hepatitis B virus (HBV) small envelope glycoprotein, an HBV middle envelope glycoprotein, or an HBV large envelope glycoprotein.

The present methods may further comprise administering to the subject a glucosidase inhibitor, an antiviral agent, or both. The glucosidase inhibitor and/or antiviral agent may be administered separately or simultaneously (for example, in a unitary composition) with the administration of the viral glycoprotein, fragment, or DNA construct. The antiviral agent may be a nucleoside analog. For example, the antiviral agent is 1-(2-fluoro-5-methyl-beta-L-arabinofuranosyl)-uracil (L-FMAU), 2-Amino-9-[(1S,3R,4S)-4-hydroxy-3-(hydroxyl)-2-methylidenecyclopentyl]-6,9-dihydro-3H-purin-6-one (Entecavir), or a combination thereof. The glucosidase inhibitor may be, for example, 6-O-butanoyl castanospermine (BuCas), a deoxynorjirmycin (e.g., N-nonyl deoxynorjirmycin), or a combination thereof.

### EXAMPLES

### In Vitro Generation of Peptide Specific Cytotoxic T Lymphocytes (CTLs)

Heparinized blood from healthy HLA-A2 donors was purchased from Research Blood Components, LLC, (Brighton, Mass.), Peripheral blood mononuclear cells were purified and cultured as described (13, 21). After initial stimulation with synthetic peptide, T cells were re- stimulated with CD4/CD8 T cell depleted autologous monocytes pulsed with synthetic peptide at 10 μg/ml for 5 days. IL-2 treatment and in vitro re-stimulation were repeated thrice prior to use of in vitro expanded T cells in ELISpot assays. The present inventors' previous work has demonstrated that T cells expanded in this manner secrete granzyme Band have surface CD8, hallmarks of the cytolytic potential of CD8+ T cells, so these cells are referred to as CTLs (21).

### ELISpot Assays

In vitro expanded CTLs were used as effectors in ELISpot assays to assess antigen stimulated interferon-γ release according to the manufacturer's instructions (BD-Pharmingen, San Jose, Calif.). Target cells were HepG2 human hepatoma cells (HBV negative; American Type Culture Collection) or HBV-containing HepG2.2.15 cells (22). Cells were treated with glucosidase inhibitor BuCas (1 mg/ml) twice at an interval of 3 days prior to use as targets in ELISpot assays, and washed before incubation with T cells. Typically, 1×10⁵ effectors (T cells) and 5×10³ targets were used (20:1). Results are presented as number of interferon-γ producing cells per 10⁶ CD8+ T cells.

### Animals and Treatments

All experimental procedures involving woodchucks were performed under protocols approved by the Cornell University Institutional Animal Care and Use Committee. Woodchucks were born to WHV-negative females in environmentally controlled laboratory animal facilities and inoculated at 3 days of age with 5 million infectious doses of a standardized WHV inoculum (23). Woodchucks were selected as chronic WHV carriers based on persistent detection of WHV surface antigen (WHsAg) and WHV DNA in serum prior to treatments. All animals were free of HCC at the beginning of the study as determined by hepatic ultrasound examination and normal serum activity of γ-glutamyl-transferase (GUT).

Twenty adult chronically infected woodchucks were stratified by age, sex, body weight, serum viral load, and serum GGT activity into four treatment groups of five animals each. Drug was administered orally at 100 mg/kg (in sterile water), chosen after an initial dose finding trial. Following a single oral dose of 100 mg/kg, the average observed Cmax was 7.7 μg/ml (range 5.0-12.1). The subunit vaccine consisted of 22-nm WHsAg particles, purified by zonal ultracentrifugation from serum of WHV7P1-infected WHV carriers (24), inactivated with formalin, and adsorbed onto alum. Prior to alum adsorption, vaccine was tested in naive, WHV- susceptible animals and no residual virus was detected. Purified WHsAg was not pretreated with enzymes that remove preS sequences.

Blood samples were obtained for WHY DNA analysis and serological testing while animals were under general anesthesia (ketamine 50 mg/kg and xylazine 5 mg/kg intramuscularly). Samples were taken prior to drug administration on the first day of treatment and at the indicated time points. Animals were weighed at bi-weekly intervals, and observed daily; no evidence of drug-related toxicity was seen.

### Serologic Assays

Serum WHV DNA was measured quantitatively by dot blot hybridization (assay sensitivity, ≧1.0×10⁷ WHV genome equivalents per ml [WHVge/ml]) (25). Serum WHsAg, antibodies to WHV core antigen (anti-WHc), and WHY surface antigen (anti-WHs) were determined with WHV-specific enzyme immunoassays (26). Serum biochemical measurements included serum GGT, alkaline phosphatase (ALP), and marker of hepatocellular injury alanine aminotransferase (ALT), aspartate aminotransferase (AST), and sorbitol dehydrogenase (SDH) (25).

### Glycan Analysis

Sample preparation for glycan analysis was performed essentially as described (27). HPLC separation was performed using the Waters Alliance HPLC system with a Waters fluorescence detector, and quantified using the Millenium Chromatography Manager (Waters Corporation, Milford, Mass.). Tri-glucosylated structures were identified by comparison to known standards (27, 28).

### PBMC Proliferation Assay

T cell responses against WHY were determined using in vitro stimulators at concentrations optimal for cultures of woodchuck PBMCs (29, 30). Stimulators consisted of native 22-nm WHsAg (2 μg/ml), recombinant WHcAg (2 μg/ml), or synthetic peptides (10 μg/ml) corresponding to either native viral sequences or predicted N-de-glycosylated sequences (Table 1, below).

TABLE 1
Peptides used in PBMC proliferation assay
Previously used peptides:

| | |
|---|---|
| S1: | MGNNIKVTFNPDKIA |
| | (SEQ ID NO: 1) |
| S7/8: | GRKPTPPTPPLRDTHPHLTM |
| | (SEQ ID NO: 2) |
| S11: | DPALSPEMSPSSLLGLLAGLQVV |
| | (SEQ ID NO: 3) |
| S12/13: | YFLWTKILTIAQNLDWWCTS |
| | (SEQ ID NO: 41) |
| S18: | YCCCLKPT AGNCTCWPIPSS |
| | (SEQ ID NO: 5) |
| S21: | LSILPPFIPIFVLFFLIWVYI |
| | (SEQ ID NO: 6) |

New peptides used in this study:

| | |
|---|---|
| PreS2-N: | LTMKNQTFHLQGFVDGLR |
| | (SEQ ID NO: 7) |
| PreS2-D: | LTMKDQTFHLQGFVDGLR |
| | (SEQ ID NO: 8) |
| S-N: | CLKPTAGNCTCWPIPSSW |
| | (SEQ ID NO: 9) |
| S-D: | CLKPTAGDCTCWPIPSSW |
| | (SEQ ID NO: 10) |

The in vitro proliferation assay using woodchuck PBMCs labeled dividing cells with [2-³H]adenine (Amersham Pharmacia Biotech, Inc., Arlington Heights, Ill.). Woodchuck PBMCs were isolated from whole blood and stimulated as described (30, 31). Counts per minute of triplicate PBMC cultures were averaged and expressed as a stimulation index (SI) by dividing the average sample counts per minute in the presence of the stimulator by that observed in the absence of stimulator (six replicates). A SI value of ≧3.1 was considered to represent a positive, specific T-cell response.

### CTLs Raised Against Aspartic Acid-Containing Envelope Peptides Recognize HBV Producing Cells

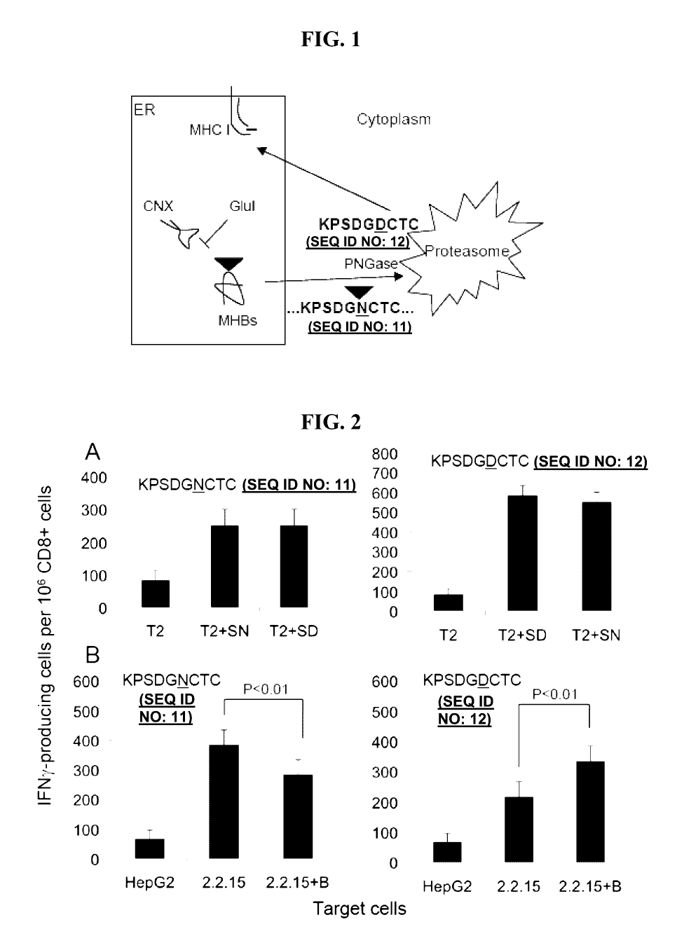

The ER chaperone calnexin (CNX) binds to nascent glycoproteins that are mono-glycosylated due to trimming of terminal glucoses by glucosidases (FIG. 1). FIG. 1 depicts interference of the interaction of MHBs with calnexin (CNX) in the ER by glucosidase inhibitor (GluI), with subsequent retrotranslocation to the cytoplasm. Both de-N-glycosylation by PNGase and degradation by the proteasotne result in the production of a novel D-peptide in place of the original N-peptide. These peptides are now available for re-import into the ER and loading into empty MHC class I complexes. The inverted triangle represents a tri-glucosylated N-glycan chain.

It was hypothesized that inhibition of glucosidases would prevent HBV MHBs interaction with CNX and cause accumulation of misfolded MHBs. Misfolded protein might be retrotranslocated from the ER to the cytoplasm, and degraded by proteasomes. Accumulation of unglycosylated MHBs when cells were treated simultaneously with proteasome inhibitors and glucosidase inhibitor suggested that de-N-glycosylation occured (5). Cellular PNGase cleaves the N-glycosidic linkage between the core N-acetylglucosamine and asparagine (N), with deamidation to aspartic acid (D). Thus, formerly N-glycosylated peptides that emerge from the proteasome will differ from peptides that were never glycosylated. Since the newly characterized “D” containing epitopes are not specified by the viral genome and presumably result from posttranslational editing, they are herein referred to as “editopes”.

Peptides presented on the surface of a cell in the context of the MHC class I complex should be recognized with high sensitivity upon incubation with cognate peptide-primed CTLs, with specific killing of the target cells. Previously, preparation of CTLs by stimulation with a known HLA-A2 restricted antigenic peptide, 183-FLLTRILTI was reported (13). This peptide represents amino acids 183-191 of LHBs_(32). Such CTLs recognized. HepG2.2.15 target cells expressing viral antigens. HepG2.2.15, and the parental, HBV-negative, hepatoblastoma cell line HepG2, express HLA-A2 class I molecules, but not HLA class II (33). The present inventors tested whether a de-N-glycosylated HBs peptide could elicit CTLs from human peripheral blood mononuclear cells (PBMCs) that recognize peptides presented by HepG2.2.15 cells. PBMCs from healthy HLA-A2 positive donors were isolated and stimulated in vitro with either amino acids 304-312 KPSDGNCTC (N-peptide, FIG. 1) (SEQ ID NO: 11), or the corresponding de-N-glycosylated KPSDGDCTC (D-peptide) (SEQ ID NO: 12). Peptides conformed to the consensus for HLAA2 binding according to the SYFPEITHI prediction algorithm (34). In vitro stimulated CTLs were incubated with either uninfected HepG2 cells or HBV-producing HepG2.2.15 cells. Target cell recognition was quantified by interferon-γ ELISpot assay.

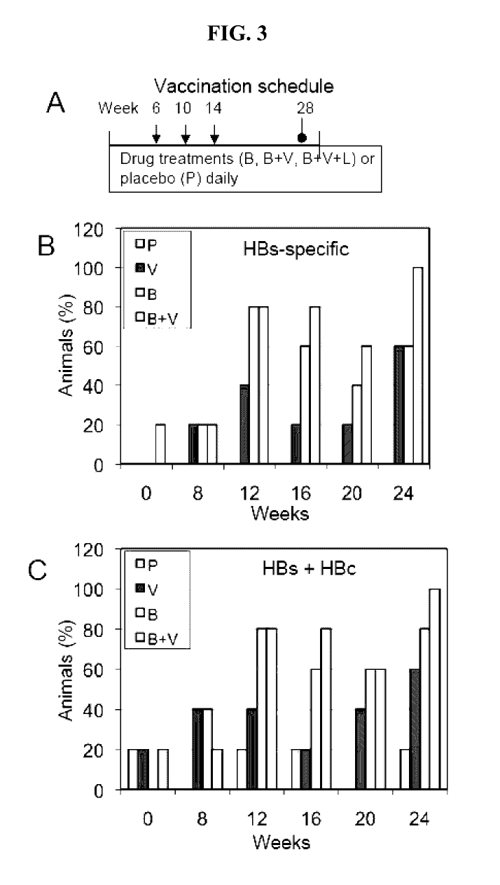

Both the natural N-peptide and the non-templated D-peptide were effective elicitors of specific CTLs that recognize HLA-A2 expressing T2 target cells, with significant cross-reactivity (FIG. 2).

As shown in FIG. 2, PBMCs isolated from healthy HLA-A2+ human donor blood were stimulated in vitro with peptides corresponding to the HLA-A2 restricted CTL epitope from HBs (KPSDGNCTC) (SEQ ID NO: 11) or the ‘D’ substituted peptide (KPSDGDCTC) (SEQ ID NO: 12). The ability of in vitro generated CTLs to recognize and secrete interferon-γ was evaluated by ELISpot assay. In FIG. 2A, CTLs generated against ‘N’ containing peptide and the corresponding ‘D’ containing peptide were incubated with T2 cells pulsed with either ‘N’ or ‘D’ containing peptide to assess T cell cross-reactivity. In FIG. 2B, HBV negative HepG2 cells or HBV positive HepG2.2.1.5 cells, either left untreated or treated with BuCas (1 mg/ml) twice for three day intervals were used as targets. Target cells (5000 cells per well) were washed once before they were co-incubated with CTLs (100,000 cells/well) in an ELISpot plate. Error bars represent SEM of experimental replicates. The P value was calculated from a Student's t-Test analysis of experimental results.

Presentation of the D-peptide epitope by target cells was increased significantly by 6-0-butanoyl-castanospermine (BuCas) treatment, presumably because de-N-glycosylated epitope production was enhanced by glucosidase inhibition. Presentation of the N-peptide epitope was reduced in cells treated with the BuCas, consistent with increased protein turnover. Similar results were obtained in an independent experiment with another donor (data not shown). BuCas-induced changes were specific for the viral envelope glycoprotein, and not seen with CTLs primed with an epitope from HBV core antigen (13). These results show that (1) D-peptides are stimulatory and (2) glucosidase inhibition increases the degree to which HepG2.2.15 cells are recognized by CTLs primed with D-peptide but not N-peptide.

### Treatment of Chronic WHV Carrier Woodchucks with Antiviral and Immunostimulatory Agents

Next, the present inventors investigated whether D-peptide-specific responses could be observed in vivo following glucosidase inhibition. Woodchuck hepatitis virus (WHY) shares DNA sequence homology and pathobiological features with human HBV. WHV establishes chronic infection in outbred woodchucks and is considered to be a model for the human virus (20). It was previously demonstrated by the present inventors that WHY MHBs is sensitive to glucosidase inhibition in vivo (11). Antigen-specific proliferative cell responses of PMBCs were examined from woodchucks chronically infected with WHV as a function of treatment with BuCas.

Woodchucks chronically infected with WHV experienced significant immunological responses to envelope proteins following immunization with WHsAg-containing vaccines, especially in the context of low viral and antigen loads following treatment with an effective antiviral agent, 1-(2-fluoro-5-methyl-beta-L-arabinofuranosyl)-uracil (L-FMAU) (29, 35). Since BuCas treatment might be expected to reduce the amount of MHBs in the circulation and/or alter its immunological profile, the response to BuCas administration along with WHsAg vaccine was investigated. Twenty-five woodchucks chronically infected with WHY were divided into five treatment groups: Placebo, Vaccine alone (V), BuCas alone (B), vaccine plus BuCas (V+B) and V+B plus L-FMAU (V+B+L). Four uninfected animals served as controls. Vaccine interventions were as shown in FIG. 3A, which depicts the scheduled treatment of woodchucks. Arrows indicate vaccination with complexes of alum and surface antigen for selected groups of animals. Circle, vaccination of animals with D-peptide. In FIG. 3B, PBMCs were isolated at the indicated time points, and cultured as described in Materials and Methods. Peptide antigens are shown in Table 1; in addition, full length WHV core and HBs were used as antigens. Animals were scored as positive if cells proliferated above the cut-off value of 2:3.1. Treatment groups are designated as P, placebo; B, BuCas; V, vaccine; B+V, BuCa plus vaccine. Percentage of animals with a positive response to one or more WHsAg-related peptides is shown. FIG. 3C, as for B, shows the percentage of animals with a positive response to the entire WHsAg, and/or WHcAg.

Viremia and antigenemia remained relatively stable in all placebo animals (Table 2, below, and data not shown). These parameters were not altered significantly by treatment with either BuCas alone or the combination of BuCas and vaccine, at all times tested; representative data are shown from week 0 (baseline) and week 10 (4 weeks after the first vaccination).

TABLE 2
Summary of key woodchuck serum parameters at weeks 0 and 10

| | WHV DNA\hat{ }, Av(range) | | WHsAg*, Av(range) | | ALT^{#}, Av(range) | | Glu3, % |
|---|---|---|---|---|---|---|---|
| Group | Week 0 | Week 10 | Week 0 | Week 10 | Week 0 | Week 10 | Week 10 |
| U | UD+ | UD | 0 | 0 | 4.8 (4-7) | 5.5 (4-8) | NT+ |
| P | 5.3 (.5-15) | 4.1 (.7-16) | .39 (.26-.55) | .41 (.26-.6) | 6.0 (5-7) | 9.6 (8-11) | NT |
| V | 8.8 (1.6-19) | 8.5 (.22-16) | .40 (.3-.47) | .44 (.32-.56) | 6.2 (4-11) | 9.8 (5-18) | 0 |
| B | 2.7 (.4-7.0) | 1.9 (.9-4.8) | .38 (.28-.43) | .42 (.33-.52) | 7.4 (4-9) | 15.6 (10-22) | .49 (0-.90) |
| V + B | 13 (1.6-50) | 13 (2.1-51) | .40 (.31-.55) | .44 (.34-.55) | 8 (4-20) | 12.6 (9-18) | .33 (0-.55) |
| V + B + L | 19 (12-29) | UD+ | .43 (.27-.53) | .24 (0-.47) | 7.2 (4-16) | 9.4 (6-13) | .53 (.41-.64) |

\hat{ }DNA level is expressed as genome equivalents (×10¹⁰)
*WHsAg level is indicated in optical density units
^{#}ALT is indicated in Units/L
+UD, <E07 GE, the detection limit of dot blot hybridization; NT. not tested

Markers of liver injury such as ALT, AST and GGT were also fairly stable (Table 2), excepting an animal in group Y+B that succumbed to hepatocellular carcinoma at about week 20. The triple combination Y+B+L resulted in marked reduction of viremia, consistent with a previous trial (29, 35). Thus, BuCas treatment was not incompatible with reduction in viral load.

In vivo, levels of circulating glycoproteins with N-glycans bearing three terminal glucose residues reflect the extent of glucosidase inhibition (11). Animals treated with BuCas were determined to have microgram per milliliter levels of the drug (Materials and Methods), which impaired glycan processing, seen as tri-glucosylated glycans in the sera of BuCas-treated animals (Table 2). Note that BuCas-treated animals that were negative for tri- glucosylated glycans at the 10-week time point were positive at one or more other time points (data not shown). No tri-glucosylated glycans were detected in any drug-naïve animals (Table 2).

Immunoblotting analysis of sera revealed visible drops in circulating MHBs in all of the animals in Y+B+L group, consistent with reductions in total surface antigen (Table 2). However, treatment with either vaccine or BuCas, alone or in combination, did not decrease MHBs levels at any time points (data not shown).

### Proliferation of PBMCs from Woodchucks Chronically Infected with WHV in Response to Viral Antigens and Pharmacologically Induced Neo-Antigens

Although reagents to dissect the immune response of woodchucks are limited, assays to measure lymphocyte recognition of specific epitopes have been implemented. PMBCs are isolated from the animals and incubated with antigen in vitro; proliferation is assumed to be evidence of antigen recognition and stimulation. PMBCs were isolated from animals at the indicated times (FIG. 3) and incubated with a panel of viral antigens, including intact WHsAg and various peptides of WHsAg (Table 1). Most the of the peptides were shown previously to induce strong proliferation of PBMCs from woodchucks with resolved WHY infections or vaccinated with WHsAg (29, 30, 35); these cells have been shown to be CD3+ T cells. The panel also included both D- and N-containing peptides spanning the two N-glycosylation sites of WHV MHBs. There was no recognition of naturally specified WHY HBs epitopes incubated with PMBCs from chronically infected woodchucks that were left untreated with either drug or vaccine at any time point (FIG. 3, group P). This is as expected, since chronically infected animals are considered tolerant and are unresponsive to HBV antigens (20).

Some vaccinated animals (Group V) produced PMBCs that recognized WHV epitopes (FIG. 3), The two responding animals at week 12 differ from those positive at week 8 (not shown), suggesting possible sampling variation, or variation in kinetics with respect to development of antibody and T cell responses. Strikingly, BuCas treatment alone resulted in proliferation in response to WHV HBs antigens (group B). BuCas plus vaccine also was potent at stimulating cellular responses (group B+V). Thus, despite the absence of detectable changes in antigenemia induced by the drug, virus-specific immune responses apparently occurred.

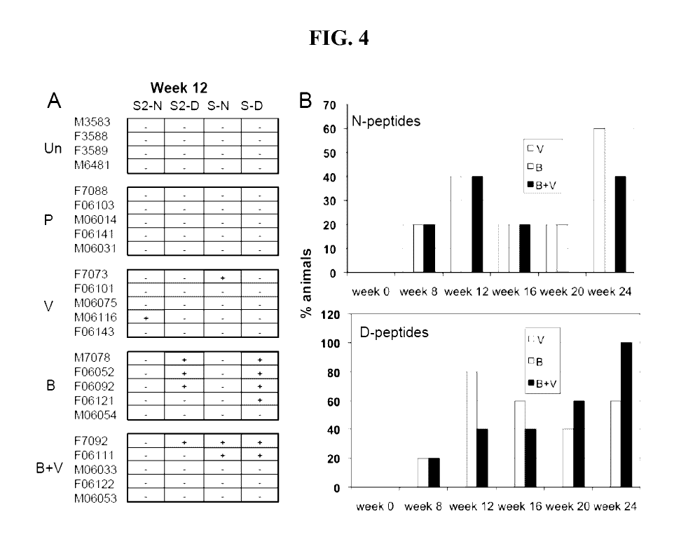

From the data in FIG. 2, a cellular immune response to D-peptide antigens was expected. Responses to the paired N/D peptides (glycosylation sequons at amino acids 4 and 146) were evaluated (FIG. 4). FIG. 4A provides detailed responses of individual animals at a single time point to N-peptides versus D-peptides. Positive response is as defined in FIG. 4. Treatment groups are designated as Un, uninfected controls; P, placebo; B, BuCas; V, vaccine; B+V, BuCa plus vaccine. FIG. 4B provides a summary of responses of groups to N-peptides and D-peptides over time.

In untreated animals, none of the peptides elicited a response. For group V, responses was restricted to N-peptides. Since the D-peptides are not specified by WHV, the lack of response is not entirely surprising. In contrast, animals in groups B and B+Y responded more strongly to D-peptides versus N-peptides. In some cases, both peptides were recognized (FIG. 4A). This response was observed as early as 8 weeks of treatment and persisted throughout (FIG. 4B).

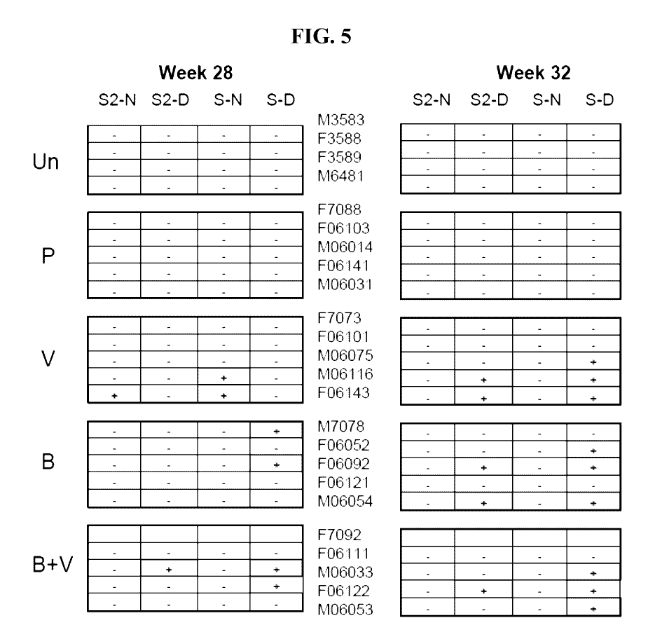

Lack of reactivity to D-peptides might be due to some animals being incapable of responding to these epitopes. To test this possibility, all animals in groups V, B, and B+V were inoculated with D-peptides in alum at week 28 (FIG. 3A). PBMCs were harvested at weeks 28 and 32, and analyzed for antigen-dependent proliferation (FIG. 5). FIG. 5 depicts the detailed responses of individual animals either pre-inoculation (week 28) or 4 weeks post- inoculation with D-peptides. Treatment groups are designated as Un, uninfected controls; P, placebo; B, BuCas; V, vaccine; B+V, BuCa plus vaccine. Woodchuck 7092 died following week 20 of the study, and thus is unscored.

Cellular responses to D-peptides were evident in all three groups at week 32 (⅗ animals positive), indicating that most animals were capable of responding to these epitopes. These data strongly suggest that D-peptides were produced and presented in animals treated with BuCas, and that these epitopes, which are herein referred to as “epitopes” are not abundantly produced in the absence of pharmacological intervention.

Normally, wild-type MHBs is very stable in cultured cells (5). However, pharmacologic inhibition of ER glucosidases that trim N-glycans on nascent proteins results destabilization of MHBs. Such treatment leads to proteasome-mediated degradation, which in turn results in increased presentation of proteasome-derived peptides by MHC class I (13). Based on the findings disclosed herein, de-N-glycosylation is expected to produce peptides in which asparagines are converted to aspartic acids (FIG. 1). The detection of D-peptides derived from MHBs presented by MHC class I on the surface of HepG2.2.15 cells treated with BuCas supported this hypothesis (FIG. 2). Thus, woodchucks chronically infected with WHV were treated with BuCas, and the effect of the drug on both viral replication and immune response to therapeutic vaccination were evaluated.

It was unexpectedly found that there was no detectable antiviral response in the drug treated woodchucks (Table 2), despite apparent efficacy in cell culture (13). Indeed, antiviral activity had previously been observed in woodchucks with a different iminocyclitol, N- nonyl deoxynojirimycin (11), There are several possible reasons for this discrepancy. First, the dose obtained with BuCas may have been insufficient to produce an antiviral effect, despite biochemical efficacy (tri-glucosylated proteins in the circulation, Table 2). Second, the two compounds do not act identically. Formation of the mono-glucosylated substrate for CNX requires sequential action of glucosidases I and II (10). Castanospertnine and its derivative BuCas are more potent inhibitors of glucosidase I than deoxynojirimycin, but the latter may have more activity against glucosidase II (36-38). Thus, more tri-glucosylated. MHBs should accumulate with BuCas. All three glucosylated species should be substrates for endomannosidase and escape from the ER (39). Finally, deoxynojirimycin prevents oligosaccharide addition some fraction of the time, but castanospermine does not (36). Secretion of MHBs is highly dependent upon the presence of N-glycan within the pre-S2 region (7).

A desirable therapeutic vaccine against chronic HBV would stimulate antiviral CTLs, which, combined with a reduction in viremia achieved by other treatments, should eliminate infected cells. Unfortunately, the response of chronically infected patients to such a vaccine was weak (40). Despite the absence of antiviral activity in the WHY infected animals, BuCas stimulated cellular immunity to viral antigen; only infected woodchucks treated with BuCas possessed PMBCs that could recognize and be primed by the D-peptides derived from MHBs. Based on the results presented herein, it is concluded that (a) D-peptide versions of the MHBs peptides can be presented by MHC class I and can activate CD8+ T cells and (b) the de- N-glycosylation can occur in vitro and in vivo following pharmacological intervention. The relatively weak response in the BuCas-treated animals to the natural N-peptides implies that there is little, if any, spontaneous generation of N-specific and that there may be limited cross recognition between cells that recognize the N- and D-epitopes.

The actual in vivo situation mechanism by which BuCas is stimulating cellular immunity is likely to be more complicated than the simplified model in FIG. 1. For instance, the limited cross recognition detected in animals is distinct from the tissue culture analysis of CD8+ CTLs from people (FIG. 2). It is unclear why non-BuCas treated HepG2.2.15 cells were recognized by D-peptide-primed CTLs. It is believed that the levels of spontaneously generated MHBs D-peptides are likely to be low, and that instead cross recognition of the N-peptide epitope by the CTLs primed with D-peptides is occurring, as was shown with exogenous peptide for the T2 cells. Some degree of cross recognition also was observed for a pair of tyrosinase peptides (41). The reason for this discrepancy is not known.

It also should be noted that the proliferative response in the woodchucks likely involves other immune cells as well as CD8+ T cells. The proliferating PBMCs include CD3+ T cells, although their CD8 status cannot be determined due to lack of specific antibody. Drug treatment might affect components of the antigen processing and presentation apparatus; unoccupied MHC class I molecules are destabilized by glucosidase inhibition (42). The WHV MHBs protein itself has been reported to suppress MHC class I presentation levels (43). Although BuCas treatment does not delectably reduce circulating MHBs, it is possible that intracellular levels are decreased, influencing formation of MHC class I complexes.

Although the human genome is estimated to contain 25,000 or fewer protein-coding genes, post-translational modifications expand protein diversity. Posttranslational editing refers to the alteration of a polypeptide sequence such that it differs from the gene from which it was specified. The enzymatic hydrolysis of N-linked glycan from the asparagines of glycoproteins by the action of the mammalian PNGase results in the conversion of the asparagines to aspartic acids. It is herein suggested that this is a form of posttranslational editing, and where it results in new epitopes, not specified by genome, which may be referred to as “editoping”.
1. Guidotti L G, Chisari F V. Immunobiology and pathogenesis of viral hepatitis. Annu. Rev. Pathol. Mech. Dis. 2006;1:23-61.
2. Rehermann B. Chronic infections with hepatotropic viruses: mechanisms of impairment of cellular immune responses. Sem. Liver Dis. 2007;27:152-160.
3. Yewdell J W, Bennink J R. Mechanisms of viral interference with MHC class I antigen processing and presentation, Annu Rev Cell Dev Biol 1999;15:579-606.
4. Bruss V. Envelopment of the hepatitis B virus nucleocapsid. Virus Res 2001;106:199-209.
5. Simsek I. Mehta A, Zhou T, Dwek R A, Block T. Hepatitis B Virus Large and Middle glycoproteins are degraded by a proteasome pathway in glucosidase-inhibited cells but not in cells with functional glucosidase enzyme. J Virol. 2005;79:12914-12920.
6. Block T M, Mehta A S, Blumberg B S, Dwek R A. Does rapid oligomerization of hepatitis B envelope proteins play a role in resistance to proteasome degradation and enhance chronicity? DNA Cell Biol 2006;25:165-170.
7. Mehta A, Lu X, Block T M, Blumberg B S, Dwek R A. Hepatitis B virus (HBV) envelope glycoproteins vary drastically in their sensitivity to glycan processing: evidence that alteration of a single N-linked glycosylation site can regulate HBV secretion. Proc Natl Acad Sci USA 1997;94:1822-1827.
8. Werr M, Prange R. Role for calnexin and N-linked glycosylation in the assembly and secretion of hepatitis B virus middle envelope protein particles. J Virol 1998;72:778-782.
9. Bergeron J J, Brenner M B, Thomas D Y, Williams DB. Calnexin: a membrane-bound chaperone of the endoplasmic reticulum. Trends Biochem Sci 1994;19:124-128.
10. Parodi A J. Protein glycosylation and its role in protein folding. Ann. Rev. Biochem. 2000;69:69-93.
11. Block T M, Lu X, Mehta A, Blumberg B S Tennant B, Ebling M, Korba B, et al. Treatment of chronic hepadnavirus infection in a woodchuck animal model with an inhibitor of protein folding and trafficking. Nature Med 1998;4:610-614.
12. Liu Y, Simsek E, Norton P, Sinnathamby G, Philip R, Block T, Zhou T, et al. The role of the downstream signal sequences in the maturation of the HBV middle surface glycoprotein; development of a novel therapeutic vaccine candidate. Virology 2007;365:10-19.
13. Simsek E, Sinnathamby G, Block T M, Liu Y, Philip R, Mehta A S, Norton P A. Inhibition of cellular alpha-glucosidases results in increased presentation of hepatitis B virus glycoprotein- derived peptides by MHC class I. Virology 2009;384:12-15.
14. Suzuki T, Seko A, Kitajima K, Inoue Y, Inoue S. Purification and enzymatic properties of peptide:N-glycanase from C3H mouse-derived L-929 fibroblast cells. Possible widespread occurrence of post-translational remodification of proteins by N-deglycosylation. Biol Chem 1994;269:17611-17618.
15. Suzuki T, Lennarz W. Hypothesis: a glycoprotein-degradation complex formed by protein-protein interaction involves cytosolic peptide:N-glycanase. Biochem Biophys Res Comm 2003;302:1-5.
16. Wiertz E, Jones T, Sun L, Bogyo M, Geuze H, Ploegh H. The human cytomegalovirus US11 gene product dislocates MHC class I heavy chains from the endoplasmic reticulum to the cytosol. Cell 1996;84:769-779.
17. Altrich-VanLith M, Ostankovitch M, Polefrone J, Mosse C, Shabanowitz J, Hunt D, Engelhard V. Processing of a class I-restricted epitope from tyrosinase requires peptide N-glycanse and the cooperative action of endoplasmic reticulum aminopeptidase 1 and clytosolic proteases. J Immunol 2006;177:5440-5450.
18. Hudrisier D, Riondi J, Mazarguil H, Gairin J E. Pleiotropic effects of post-translational modifications on the fate of viral glycoproteins a cytotoxic T cell epitopes. J Biol Chem 2001;276:38255-38260.
19. Selby M, Erickson A, Dong C, Cooper S, Parham P, Houghton M, Walker C. Hepatitis C virus envelope glycoprotein El originates in the endoplasmic reticulum and requires cytoplasmic processing for presentation by class I MHC molecules. J Immunol 1999;162:669-676.
20. Menne S, Cote P. The woodchuck as an animal model for pathogenesis and therapy of chronic hepatitis B vines infection. World J Gastroenterol 2007;13:104-124.
21. Sinnathamby G, Lauer P, Zerfass J, Hanson B, Karabudak A, Krakover Secord A A, et al. Priming and activation of human ovarian and breast cancer-specific CD8+ T cells by polyvalent Listeria monocytogenes-based vaccines. J Immunother 2009;32:856-869.
22. Sells M A, Chen, M. L., Acs, G. Hep G2 cells transfected with cloned hepatitis B virus DNA. Proc. Natl. Acad. Sci. USA 1987;84:1005-1009.
23. Cote P J, Korba B E, Miller R H, Jacob J R, Baldwin B H, Hornbuckle W E, Purcell R H, et al. Effects of age and viral determinants on chronicity as an outcome of experimental woodchuck hepatitis virus infection. Hepatol 2000;31:190-200.
24. Gerin J, Faust R, Holland P. Biophysical characterization of the adr subtype of hepatitis B antigen and preparation of anti-r sera in rabbits, J Immunol. 1975;115:100-105.
25. Menne S, Cote P J, Butler S D, George A L, Tochkov I A, Zhu Y, Xiong S, et al. Antiviral effect of orally administered lamivudine, emtricitabine, adefovir dipivoxil, and tenofovir disoproxil fumarate, alone and in combination in woodchucks with chronic woodchuck hepatitis virus infection. Antimicrob Agents Chemother 2008;52:3617-3632.
26. Cote P J, Roneker C, Cass K, Schadel F, Peterson D, Tennant B C, De Noronha F, et al. New enzyme immunoassays for the serologic detection of woodchuck hepatitis virus infection. Viral Immunol 1993;6:161-169.
27. Comunale M A, Lowman M, Long R E, Krakover J, Philip R, Seeholzer S, Evans, A A, Hann H W L, Block T M, Mehta A S. Proteomic analysis of serum associated fucosylated glycoproteins in the development of primary hepatocellular carcinoma. J Proteome Res 2006;6:308-315.
28. Royle L, Mattu T S, HartE, Langridge J I, Merry A H, Murphy N, Harvey D J, et al. An analytical and structural database provides a strategy for sequencing 0-glycans from microgram quantities of glycoproteins. Anal Biochem 2002;304:70-90.
29. Menne S, Roneker C A, Tennant B C, Korba B E, Gerin J L, Cote P J. Immunization with surface antigen vaccine alone and after treatment with 1-(2-fluoro-5-methyl-beta-L-arabinofuranosyl)-uracil (L-FMAU) breaks humoral and cell-mediated immune tolerance in chronic woodchuck hepatitis virus infection. J Virol. 2002;76:5305-5314.
30. Menne S, Tennant B C, Gerin J L, Cote P J. Chemoimmunotherapy of chronic hepatitis B virus infection in the woodchuck model overcomes immunologic tolerance and restores T-cell responses to pre-S and S regions of the viral envelope protein. J Virol 2007;81:10614-10624.
31. Menne S, Roneker C A, Roggendorf M, Gerin J L, Cote P J, Tennant B C. Deficiencies in the acute-phase cell-mediated immune response to viral antigens are associated with development of chronic woodchuck hepatitis virus infection following neonatal inoculation. J Virol 2002;76:1769-1780.
32. Chisari F V, Ferrari C. Hepatitis B virus immunopathogenesis. Annu Rev Immunol 1995;13:29-60.
33. Ito Y, Kakumu S, Yoshioka K, Wakita T, Ishikawa I, Koike K. Cytotoxic T lymphocyte activity to hepatitis B virus DNA-transfected HepG2 cells in patients with chronic hepatitis B. Gastroenterol Jpn 1993;28:657-665.
34. Rammensee H, Bachmann J, Emmerich N P, Bachor O A, Stevanovic S. SYFPEITHI: database for MHC ligands and peptide motifs. Immunogenetics 1999;50:213-219.
35. Menne S, Roneker C A, Tennant B C, Korba B E, Gerin J L, Cote P J. Immunogenic effects of woodchuck hepatitis virus surface antigen vaccine in combination with antiviral therapy: breaking of humoral and cellular immune tolerance in chronic woodchuck hepatitis virus infection. Intervirology 2002;45:237-250.
36. Gross V, Tran-Thi T A, Schwarz R T, Elbein A D, Decker K, Heinrich P C. Different effects of the glucosidase inhibitors 1-deoxynojirimycin, N-methyl-1-deoxynonrimycin and castanospermine on the glycosylation of rat alpha 1-proteinase inhibitor and alpha 1-acid glycoprotein. Biochem J 1986;236:853-860.
37. Kaushal G P, Pan Y T, Tropea J E, Mitchell M, Liu P, Elbein A D. Selective inhibition of glycoprotein-processing enzymes. Differential inhibition of glucosidases I and II in cell culture. J Biol Chem 1988;263:17278-17283.
38. Taylor D L, Kang M S, Brennan T M, Bridges C G, Sunkara P S, Tyms A S. Inhibition of alpha-glucosidase I of the glycoprotein-processing enzymes by 6-O-butanoyl castanospermine (MDL 28,574) and its consequences in human immunodeficiency virus-infected T cells. Antimicrob Agents Chemother 1994;38:1780-1787,
39. Moore S E H, Spiro R G. Demonstration that golgi endo-a-D-mannosidase provides a glucosidase-independent pathway for the formation of complex N-linked oligosaccharides of glycoproteins. J Biol Chem 1990;265:13104-13112.
40. Heathcote J, McHutchinson J, LeeS, Tong M, Benner K, Minuk G, Wright T, et al. A pilot study of the CY-1899 T-cell vaccine in subjects chronically infected with hepatitis B virus. The CY1899 T Cell Vaccine Study Group, Hepatol1999;30:531-536.
41. Mitchell M S. Phase I trial of adoptive immunotherapy with cytolytic T lymphocytes immunized against a tyrosinase epitope—In Reply. J Clin Oncol 2002;20:3176-3184.
42. Moore S E, Spiro R G. Inhibition of glucose trimming by castanospermine results in rapid degradation of unassembled major histocompatibilty complex class I molecules. J Biol Chem 1993;268:3809-3812.
43. Wang J, Michalak T I. Inhibition by woodchuck hepatitis virus of class I major histocompatibility complex presentation on hepatocytes is mediated by virus envelope pre-S2 protein and can be reversed by treatment with gamma interferon. J Virol2006;80:8541-8553.

What is claimed:

1. A method for treating a subject having a viral infection comprising:
administering to said subject a composition comprising
a viral glycoprotein or a fragment thereof,
or,
a DNA construct encoding for said viral glycoprotein or fragment thereof, wherein said glycoprotein or fragment comprises a glycosylation sequon that includes a non-templated aspartic acid residue; and,
a glucosidase inhibitor.

2. The method according to claim 1 wherein said glycoprotein is an envelope protein.

3. The method according to claim 2 wherein said glycoprotein is an HBV small envelope glycoprotein, an HBV middle envelope glycoprotein, or an HBV large envelope glycoprotein.

4. The method according to claim 1 further comprising administering to said subject a glucosidase inhibitor, an antiviral agent, or both.

5. The method according to claim 4 wherein said antiviral agent is a nucleoside analog.

6. The method according to claim 5 wherein said antiviral agent is 1-(2-fluoro-5-methyl- beta-L-arabinofuranosyl)-uracil, or 2-Amino-9-[(1S,3R,4S)-4-hydroxy-3-(hydroxymethyl)-2-methylidenecyclopentyl]-6,9-dihydro-3H-purin-6-one.

7. The method according to claim 1 wherein said glucosidase inhibitor is 6-0-butanoyl castanospermine or a deoxynorjirmycin.

8. The method according to claim 1 wherein said subject is infected with an enveloped virus.

9. The method according to claim 1 wherein said virus is hepatitis B or hepatitis C.

10. A composition comprising
a viral glycoprotein or a fragment thereof, or, a DNA construct encoding for said viral glycoprotein or fragment thereof;
a glucosidase inhibitor;
and,
a pharmaceutically acceptable carrier,
wherein said glycoprotein or fragment comprises a glycosylation sequon that includes a non-templated aspartic acid residue.

11. The composition according to claim 10 wherein said glycoprotein is an envelope protein.

12. The composition according to claim 12 wherein said glycoprotein is an HBV small envelope glycoprotein, an HBV middle envelope glycoprotein, or an HBV large envelope glycoprotein.

13. The composition according to claim 10 further comprising an antiviral agent.

14. The composition according to claim 14 wherein said antiviral agent is a nucleoside analog.

15. The composition according to claim 15 wherein said antiviral agent is 1-(2-fluoro-5-methyl-beta-L-arabinofuranosyl)-uracil, or 2-Amino-9-[(1S,3R,4S)-4-hydroxy-3-(hydroxymethyl)-2-methylidenecyclopentyl]-6,9-dihydro-3H-purin-6-one.

16. The composition according to claim 14 wherein said glucosidase inhibitor is 6-O-butanoyl castanospermine or a deoxynorjirmycin.

17. The composition according to claim 10 wherein said viral glycoprotein is of the hepatitis B virus or the hepatitis C virus.

18. The method according to claim 1 comprising administering to said subject a fragment of a viral glycoprotein having the amino acid sequence KPSDGNCTC (SEQ ID NO:11), or having the amino acid sequence KPSDGDCTC (SEQ ID NO:12).

19. The composition according to claim 10 that includes a fragment of a viral glycoprotein having the amino acid sequence KPSDGNCTC (SEQ ID NO:11), or a fragment of a viral glycoprotein having the amino acid sequence KPSDGDCTC (SEQ NO:12).
